(19) United States
(12) Patent Application Publication
(10) Pub. No.: US 2015/0148876 A1
(43) Pub. Date: May 28, 2015

(54) IMPLANTABLE DEVICE WITH AN INSULATING LAYER AND METHOD

(71) Applicant: Heraeus Precious Metals GmbH & Co. KG, Hanau (DE)

(72) Inventors: Jeremy Glynn, Buffalo, MN (US); Steve Harein, Mahtomedi, MN (US); Andreas Reisinger, Alzenau (DE); Goran Pavlovic, Schaafheim (DE)

(21) Appl. No.: 14/352,618

(22) PCT Filed: Oct. 16, 2012
(86) PCT No.: PCT/EP2012/004317

§ 371 Date: Aug. 19, 2014

(30) Foreign Application Priority Data
Oct. 19, 2011 (DE) 102011116289.9

(51) Int. Cl. A61N 1/375; H05K 7/02; B05D 3/10; B05D 1/02; B05D 1/18

(52) U.S. Cl. CPC A61N 1/375; B05D 1/02; B05D 1/18; B05D 3/10; H05K 7/02
USPC 607/116; 361/746; 29/592.1

(57) ABSTRACT

A device (100), comprising:
a housing (110) having an inner surface (160) and an outer surface (170);
an electronic unit (130, 140, 180);
   whereby the housing (110) surrounds the electronic unit (180) at least in part;
   whereby at least a part of the inner surface (160) of the housing (110) comprises an electrically insulating coating (120) that contains at least 30 wt.-% of a polymer and has a coating surface (150) facing the inner surface (160);
   whereby the inner surface (160) and the coating surface (150) are interconnected.

The invention relates to the field of implantable devices such as pacemakers or defibrillators, and their various components. In particular, the invention relates to an implantable device with a housing liner which is configured to have particularly high dielectric strength. The invention further relates to a method for providing such a device and a method for using such a device.

Implantable devices for various applications are known from the prior art. Accordingly, besides the defibrillators and cardiac pacemakers which can already be used for therapy, such as described in the publication “Herzschrittmacher-und Defibrillator-Therapie: Indikation—Program-mierung—Nachsorge, ed. Gerd Fröhlig et al.; Thieme Verlag, Stuttgart, 2005; ISBN 9783131171818, diagnostic devices that can be implanted are available as well. It is common to these devices is that they have both electronic components and mechanical components. Since the devices are placed in the body of a user, for example, the electronic components, at least their sensitive components, should be shielded against body fluid.

Since medical devices are usually fully implanted, they typically comprise a battery for their supply of electrical power. However, it is also conceivable, to transmit electrical energy through induction coils to the implantable device. It is definitely useful and often necessary firstly to protect the electronic components against moisture and the body from unwanted electrical currents. In order to ensure this insulation, there is usually an insulating insert between the housing of the device and the electronic components.

This insulating liner is currently glued-in by hand, for example in cardiac pacemakers or defibrillators (ICD—implantable cardioverter defibrillator). This is very costly and cumbersome. There is also the risk that the film is not glued-in properly or that it slips while it is being glued-in and so fails its essential purpose of shielding. In addition, bubbles situated between the housing and the liner can produce electrical bridges. As a result, glued-in inserts allow a dielectric strength of less than 2 kV to be attained.

In particular, when the implantable devices have an electronic component or an electronic subassembly that develops elevated voltages, such as defibrillators, it is important that adequate insulation is ensured to exist between the component or sub-assembly and the housing of the implantable device contacting the body housing. Otherwise, inadvertent electric shocks or discharges with serious consequences can occur in the body containing the implantable device.

It is therefore an object of the invention to overcome, at least partially, at least one of the disadvantages resulting according to the prior art.

It is a further object of the invention to provide a device which enables the maximal possible protection and safety of the user, while meeting the requirement for implantable medical devices.

Further, it is an object to be able to generate a reproducible and adequate long-term insulation of the electronic components in said device, in particular to provide for equal or better dielectric strength of the device while the requisite coating takes up less space.

A further object is to improve the dielectric strengths of the housing to protect the user and the electrical components, in particular the high-voltage components in an implant. Another object is to optimise a process for producing said device, in that it allows for an inexpensive and reproducible way to manufacture the devices.

A contribution to meeting at least one of the preceding objects is made by the invention having the features of the independent claims. Advantageous refinements of the invention, which can be implemented alone or in any combination, are specified in the dependent patent claims.

In a first aspect, the invention relates to a device, comprising:
a housing having an inner surface and an outer surface;
an electronic unit;
   whereby the housing surrounds the electronic unit at least in part; whereby at least a part of the inner surface of the housing comprises an electrically insulating coating that contains at least 30 wt.-% of a polymer and has a coating surface facing the inner surface; whereby the inner surface and the coating surface are interconnected.

The device can serve different purposes. Preferably it is a medical device, in particular an implantable medical device. A medical device shall be understood, in particular, to be a device that has a medical function, such as for example a therapeutic, a diagnostic or a surgical function. An implantable medical device shall be understood to mean a medical device that can be introduced, at least in part, into the body of a user. Moreover, it should assume a medical function, such as, for example, a diagnostic, surgical or therapeutic function. For this purpose, the device can have a particular configuration, such as, for example, a particular shape in order to disturb the user wearing it as little as possible. Furthermore, the implantable device can be configured to disturb and impact the body of the user as little as possible when the device is being inserted and carried. This can be achieved, for example, in that the device comprises, for example, a rounded outer shape and in that the surface contacting the body of the user is made, for example, from a bio-compatible material.

The devices according to the invention can also be configured as “active implantable medical device” (AIMD) and particularly preferably as a therapeutic device. In particular, the medical function can comprise at least one actuator function, in which at least one actuator is used to exert at least one stimulus on the body tissue, in particular an electrical stimulus.

As a matter of principle, the term, active implantable medical device—also called AIMD—shall comprise all implantable medical devices that can conduct electrical signals from a, in particular, hermetically sealed housing to a part of the body tissue of the user and/or receive electrical signals from the part of the body tissue of the user. Accordingly, the term, active implantable medical device, comprises, in particular, cardiac pacemakers, cochlea implants, implantable cardioverters/defibrillators (ICD), nerve, brain, organ or muscle stimulators as well as implantable monitoring devices, hearing aids, retinal implants, implantable drug pumps, artificial hearts, bone growth stimulators, prostate implants, stomach implants or the like.

The shape and dimensions of the housing of the device should be selected appropriately such that it does not hinder the user upon implantation, of at least a portion of the device. Further, the housing should have an appropriate shape such that the components of the device, such as battery, controller, capacitor and/or cable can be accommodated in optimal space-saving manner. If this concerns a device that is fully inserted into the body, such as a therapeutic or diagnostic device, in particular an ICD, the volume surrounded by the housing should be in a range of 0.1 to 50 cm³, preferably in a range of 0.5 to 30 cm³, more preferably in a range of 5 to 20 cm³, particularly preferably in a range of 5 to 10 cm³. In this context, the width and length of the housing of the device can each be in a range of 1 to 10 cm, preferably in a range from 3 to 7 cm. The height is often in the range of 0.4 to 2 cm and preferably in a range of 0.5 to 1.5 cm. The shape of the housing can be arbitrary. For example, the shape can be angular, round, oval or conical. Preferably, the housing of the device comprises no sharp edges and corners. The housing can consist of one or more parts. Preferably, the housing consists of two shell-like parts. The housing should be adapted to surround the remaining components of the device at least partially. The housing can have one or more openings allowing components to be passed into the housing. For example, a cable can be passed from the inside of the housing through an opening of the housing to the outside. Preferably, the housing is hermetically sealed with respect to the outside, at least when the device is in use. Accordingly, the housing parts also take up the bushings of the contact that is electrically connected to the heart muscle.

According to the invention, the housing has an inner and an outer surface. In this context, the material of the inner and outer surfaces need not be the same, but it can. The inner and outer surfaces can be interconnected. In the state of the device being in use, the inner surface faces the other components of the device. The external surface faces towards the body of the user following implantation and can contact the body, at least in part. In the non-implanted state, the outer surface faces away from the components of the device.

The device according to the invention further comprises an electronic unit. Said electronic unit can consist of one or more electronic components that are capable of generating, storing, conducting or consuming electric charge. For example, the electronic unit can be selected from the group consisting of a battery, a capacitor, a control unit and a cable or a combination of at least two thereof. According to the invention, the housing surrounds the electronic unit at least in part. Accordingly, for example one part of a battery or other electronic component can be arranged within the housing, and another part of said component can be arranged outside the housing. Alternatively or additionally one or more further electronic components can be partially or fully arranged within the housing. Preferably, the electronic unit is arranged fully within the housing, i.e. surrounded by the inner surface.

According to the invention, at least a part of the inner surface of the housing comprises an electrically insulating coating that contains at least 30 wt.-%, preferably at least 40 wt.-%, particularly preferably at least 50 wt.-% of a polymer. In turn, the electrically insulating coating comprises a coating surface that faces the inner surface of the housing. According to the invention, the inner surface of the housing and the coating surface are interconnected. According to the invention, a coating shall be understood to be a layer or a film that extends over the connected area. According to the invention, the coating surface is the surface of the coating that faces the inner surface of the housing and contacts the inner surface of the housing.

According to the invention, connecting shall be understood to mean that the part of the coating surface and the part of the inner surface of the housing to be connected contact each other directly or indirectly, whereby direct contact is preferable. Direct contact shall be understood to mean that the coating surface is immediately adjacent to the inner surface of the housing. This is the case, in particular, when no adhesion-promoting substances or layers between electrically insulating coating and inner surface are used. Conversely, in the case of indirect contact of coating surface and inner surface of the housing, one or more intermediate layers, preferably adhesion promoters such as adhesives or waxes, are provided between the coating surface and the inner surface.

The contacting of the surfaces be implemented through any kind of contacting two surfaces. The contacting is preferably effected by the application of the coating, preferably in the form of a liquid phase, to the inner surface of the housing. According to the invention, all materials capable of flowing are referred to as liquid phase. Examples of which include liquid solutions, such as polymers dissolved in a solvent, or mixtures of liquids, for example, a substantially solvent-free varnish with monomer cross-linker and initiator, in which at least two chemical substances result in a homogeneous liquid mixture, or dispersions, in which at least two substances result in a heterogeneous mixture, or powders of a wide variety of compositions. The liquid phase can be applied either by depositing the liquid phase on the inner surface or by dipping the inner surface into the liquid phase. Depositing can, for example, mean brushing, rolling, spraying, printing or injecting of the coating in the form of the liquid phase to the inner surface of the housing. Preferably, the coating change its state after the inner surface of the housing is contacted to the liquid phase. This change of state causes the liquid phase to turn into a solid phase in the form of the electrically insulating coating. In this context, the coating forms a stable, in particular film-like, connection to the housing.

A change of state shall be understood, for example, to be an affixing of components of the liquid phase during the transition to the electrically insulating coating on the inner surface of the housing. If, for example, the liquid phase is applied in the form of a powder to the inner surface of the housing, thermal treatment of the powder can cause the ingredients of the powder to melt and fuse and to form a two-dimensional layer structure on the on the inner surface of the housing since it is affixed thereto. During the formation of the layer structure, the coating surface becomes connected to the inner surface of the housing, such that a stable unit of housing and electrically insulating coating is formed. This is often referred to as varnish.

Alternatively or additionally, the coating can be applied in the form of a solution or dispersion, whereby, after the solution or dispersion dries, a stable, in particular film-like, connection of the housing to the coating is formed as well. Again, the components of the electrically insulating coating are fixed in place on the inner surface of the housing after the coating dries. During or after drying, a chemical reaction of components of the electrically insulating coating with each other or with the inner surface of the housing can proceed.

As already mentioned, the connecting can proceed, for example, through a chemical reaction of the components of the liquid phase or of the electrically insulating coating and components of the inner surface of the housing. Based on functional groups, the chemical reaction can proceed either on the coating surface or on the inner surface of the housing or both. In this context, functional groups of the coating surface can react with components of the inner surface of the housing and vice versa. Conceivable as functional group are, for example, groups of molecules, which easily enter into a reaction, such as hydrophilic groups. The functional groups can preferably be selected from the group consisting of double bonds, in particular vinyl groups (R₂C═CH—), allyl groups (R₂C═CHCR₂), alkinyl groups (RC≡C—), hydroxyl groups (—OH), sulfhydryl groups (—SH, —SH₂), ester groups (—COOR), acid groups (—COOH), ether groups (—CHOR), amine groups (—NH₂, —NHR, NR₂), epoxy group (—COC) and phosphate groups (—PO₃OH) or combination thereof. The strength of the connection of the surface coating to the inner surface of the housing can be varied by choice of the type and/or number of functional groups. The reaction of the coating surface and the inner surface can be initiated or accelerated by various measures after contacting of the two surfaces. The measure can, for example, be selected from the group consisting of elevated temperature, preferably in a range of 60 to 120° C., convection, light, in particular IR or UV light, and pressure or a combination of at least two thereof.

In addition, the connection of the surface coating and the inner surface of the housing can be implemented via a physical interaction of the coating surface and the inner surface of the housing. For example, the coating can penetrate at least in part into cavities of the inner surface of the housing when the coating contacts the inner surface of the housing. This can result in a very strong connection, which is stronger than, for example, an adhesive bonding of two surfaces. The physical interaction can be effected, for example, in that the electrically insulating coating is contacted to the inner surface of the housing in the form of a liquid solution, a dispersion or as a powder. The liquid phase can have sufficiently low viscosity and/or the particle size of the powder can be sufficiently small such that either can penetrate into the cavities of the inner surface of the housing and then become solidified. The solidification can be attained in different ways. For example, the liquid phase can have a solvent that is volatile and leaves behind the solid constituents of the liquid phase after it is evaporated. An example is the formation of a varnish. Alternatively or additionally, a chemical reaction can be triggered after the liquid phase is applied to the inner surface of the housing and can cause the coating, for example in the form of a varnish, to cure while covering the surface. For example, cross-linking of a portion of the polymer to each other or to other components of the coating or to components of the inner surface can proceed when the inner surface of the housing is connected to the electrically insulating coating.

In a preferred embodiment, the electronic unit contains a capacitor. Particularly preferably, the device according to the invention is a cardiac pacemaker or an implantable cardioverter—defibrillator (ICD), either a ventricular or an atrio-ventricular ICD. A cardiac pacemaker or ICD contains, aside from the capacitor, at least battery and an electrode. The electrode can serve both for receiving signals from the surrounding tissue as well as for transmitting electrical impulses that are generated in the battery and/or capacitor or both.

Preferably, the capacitor has a capacity in a range of 50 to 1,000 μF, more preferably in a range of 100 to 800 μF, particularly preferably in a range of 200 to 500 μF. These capacities are sufficient to operate a conventional ICD as described in the publication “Herzschrittmacher-und Defibrillator-Therapie: Indikation—Programmierung—Nachsorge”, ed. Gerd Fröhlig et al.; Thieme Verlag, Stuttgart, 2005; ISBN 9783131171818. This allows, for example, an electric shock having a shock effect of 30 joules, which it transmits to the electrode and therefore to the heart, to be attained. The capacitor can be designed, for example, such that it can emit current pulses of a voltage in a range of 500 to 1,000 V, preferably in a range of 600 to 900 V, particularly preferably in a range of 750 to 800 V.

The housing of the device of the invention can include any, especially conductive, material. Preferably, the housing is made of a material which is not rejected by the body, into which it is to be implanted or irritates or affects said body. Further, the material of the housing should be sufficiently stable so as not to be damaged by the forces acting during insertion of the device into the body. Furthermore, stringent requirements exist regarding the corrosion resistance of the material. Thus, the residence time of the device of the invention in the body is increased. Preferably, the housing is only slightly deformable in order to protect the content of the device from external forces while the device is in use. Electrically conductive materials and electrically non- or slightly conductive materials are suitable for this purpose. The electrically conductive materials include, for example, metals and electrically conductive polymers. The electrically non-conductive or slightly conductive materials include, for example, glass, ceramics or electrical insulating polymers. Preferably, the material of the housing includes a metal. Particularly preferably, the material of the housing is a metal, in particular a metal selected from the group consisting of platinum, titanium, iron, and alloys containing these. In a preferred embodiment of the device, the housing contains at least 40 wt.-%, more preferably at least 70 wt.-%, particularly preferably at least 90 wt.-% titanium, relative to the housing. The remaining max. 60 wt.-% can be selected from the group consisting of aluminium, vanadium, niobium and a polymer and a combination of at least two thereof. Preferably this concerns an alloy selected from the group consisting of: grade 1 titanium, grade 23 titanium, grade 2 titanium, particularly preferably an alloy with a titanium content of more than 80 wt.-%, relative to the housing. The alloy can further be a Ti6Al4V alloy, whereby the aluminium content is preferably 6 wt.-% and the vanadium content is preferably 4 wt.-%, relative to the alloy. In a further preferred embodiment, a material can be used for the housing, which contains at least 50 weight-%, preferably at least 60 wt.-%, and particularly preferably at least 70 wt.-% of iron, and in a range of 15 to 30 wt.-%, preferably in a range of 17 to 28 wt.-%, and particularly preferably in a range of 20 to 27 wt.-% of alloy metals other than iron, whereby the sum of the weight-% specifications in each case adds up to 100. The housing can further comprise a material that contains more than 50 wt.-%, preferably more than 60 wt.-%, particularly preferably more than 70 wt.-% of iron, relative to the housing. Moreover, the housing can contains materials selected from the group consisting of chromium, nickel, magnesium, silicon and carbon. Preferably this concerns an alloy selected from the group of stainless steels SS 304L and SS316L.

The polymer contained in the coating can be any polymer which is suitable for stabilising the connection of the inner surface of the housing and the coating surface. In addition, the polymer can assume the function to keep the other components of the electrically insulating coating together. The polymer can be designed to be thermosetting or thermoplastic. Thermosetting polymers generally comprise a certain degree of cross-linking between the polymer chains, whereas thermoplastic polymers have no or only a very little cross-linking among the polymer chains.

All polymers that are solid at room temperature and/or body temperature can be used as polymer. Preferably, the electrically insulating coating contains synthetic polymers, in particular selected from the group consisting of polyethylene (PE), polypropylene (PP), polyvinyl chloride (PVC), polyester, such as polycarbonates (PC) or polyethylene terephthalate (PET), polystyrene (PS), polytetrafluoroethylene (PTFE), polymethyl methacrylate (PMMA), polyamides, polyimides, polyethylene glycol (PEG) and silicon or combinations thereof. In a preferred embodiment of the device, the polymer is selected from the group consisting of acrylates, alkyd resin, polyester imide, polyamide-imide and silicones or at least two thereof. In an embodiment of the invention, the polyamide-imide or polyester imide can be imidised by more than 70 wt.-%, particularly preferably more than 85 wt.-%.

Other components of the coating can be made of any material. Preferably, these materials are of an electrically insulating type. For example, the further components of the coating can contain binding agents, pigments or further additives. The further components can be selected from the group consisting of glass, ceramics, polymer, organic dyes, inorganic dyes and carbon black, and at least two thereof. The coating has a main function, i.e. to electrically insulate the electronic components with respect to the housing and its surroundings.

In a preferred embodiment of the device, the coating has a thickness in a range of 1 to 100 μm, preferably in a range of 10 to 80 μm, particularly preferably in a range of 30 to 60 μm. To generate a coating having such dimensions, it is preferred to produce the coating, as mentioned above, by contacting a liquid phase to the inner surface of the housing, as will be described later in detail for the manufacturing method.

Preferably, the device has a volume in a range of 3 to 30 cm³, more preferably in a range of 5 to 25 cm³, particularly preferably in a range of 10 to 20 cm³. In order to be able to provide as much room as possible for the components of the device, the major part of the volume of the device is hollow. The hollow space thus obtained preferably has approximately the same volume as the entire device. The hollow space of the device is preferably formed such that a single hollow space is formed, in which the components of the device can be accommodated as space-saving as possible. Alternatively, a plurality of hollow spaces can be formed in the device just as well.

A device, in which the surface area of the insulating coating is in a range of 1 to 30 cm², preferably in a range of 5 to 25 cm² and particularly preferably in a range of 10 to 20 cm², is also preferred. Preferably, the entire inner surface of the housing is connected to the insulating coating. In addition, at least a portion of the outer surface can also be connected to an electrically insulating coating. This coating can be the same as the one on the inner surface of the housing.

In a preferred embodiment of the device, the breakdown voltage of the insulating coating is 2 kV and more, preferably 4 kV and more and particularly preferably 7 kV and more. The breakdown voltage is a measure of the insulation of the housing, i.e. including the further components of the device, with respect to the surroundings of the housing. The breakdown voltage indicates from which voltage, applied to the housing of the device, current will be conducted through the housing and the insulation coating into the interior of the device, and vice versa. For devices to be implanted in a body, the breakdown voltage of the housing of the device should be as high as possible in order both to protect the body against accidental electric shock, but also to protect the components of the device against external interference. The level of the breakdown voltage can be affected by the composition and thickness of the electrically insulating coating. If the electrically insulating coating has a high proportion of electrically insulating components such as electrically insulating polymers or other electrically insulating components, the breakdown voltage can be very high. If the connection between the inner surface of the housing and the coating surface is very stable and uniform, the layer can be made to be thinner for the same breakdown voltage than if the connection is not uniform.

Another aspect of the invention describes a method for manufacturing a device comprising the steps of:
a. providing a housing having an inner surface and an outer surface;
b. applying an electrically insulating coating that contains at least 30 wt.-% polymer and is made of a liquid phase to at least a part of the inner surface;
c. introducing an electronic unit into the housing, whereby the housing surrounds the electronic unit at least in part.

All variants of the embodiments of the device according to the invention are also applicable to the method according to the invention for manufacturing a device. The housing of the device, can comprise the same materials, shapes and characteristics, as described above for the device according to the invention. The housing has an inner and an outer surface that can be designed in the manner described above for the device. The housing can be provided in a variety of ways. The housing can, for example, be clamped by its outer surface into a frame or by held a stand such as to be fixed in space. Alternatively or additionally, the housing can be located on a movable carrier when it is provided. Preferably, the housing is provided in appropriate manner such that the inner surface is freely accessible. According to the invention, freely accessible shall be understood to mean that the entire inner surface is accessible to a means for applying the electrically insulating coating. In particular, the inner surface should be reachable by the means for application of the electrically insulating coating, in particular, it should be of a contacting type.

The application of the electrically insulating coating takes place, according to the invention, from a liquid phase, for example in the form of a liquid solution or a dispersion, to at least a part of the inner surface of the housing. The liquid phase can contain solid components. Alternatively or additionally, the liquid phase can just as well be an emulsion or dispersion. As mentioned above with reference to the device, the liquid phase can consist of multiple components. The liquid phase contains a polymer or a polymer mixture. The composition and properties of the polymer can be the same as described with reference to the device. The liquid phase preferably contains a solvent that can be selected from the group consisting of water or organic solvents or a combination thereof. The organic solvent is preferably selected from the group consisting of ether, alcohol, hydrocarbons, and acetone or a mixture of at least two thereof. In addition to the polymer, the liquid phase can also comprise solid, in particular powdery, components. The solid components can, for example, be binding agents, carbon black or pigments.

The liquid phase can be applied to the inner surface in any manner that is adapted to form layers that are as thin as possible and have a layer thickness distribution on surfaces that is as precise as possible. Two-dimensional layers having a thickness in a range of 0.1 μm to 500 μm, preferably in a range of 1 μm to 200 μm, particularly preferably in a range of 10 μm to 100 μm, can be called thin layers. In a preferred embodiment of the method, the application of the electrically insulating coating proceeds by a process selected from the group consisting of deposition or dipping or a combination thereof.

According to the invention, deposition shall be understood to mean that the liquid phase is deposited on the surface by a means. This can be done by different means. The liquid phase can, for example, be sprayed or injected through a nozzle or a valve to the surface for deposition. Alternatively or additionally, the liquid phase can, for example, be applied and/or printed by means of a roll or roller. Known spraying or injection methods include, for example, micro-dosing or ink-jet printing through an opening such as a nozzle and/or a valve. Pressure can be applied to the liquid phase or the liquid phase is applied to the surface by means of gravity by dripping through an opening. Preferably, the liquid phase is deposited on the surface under pressure, for example in the form of a liquid varnish.

For example a piezo valve or a pneumatic valve, such as those known for use in ink-jet printers, can be used as nozzle or valve. These valves are capable of forming portions of the liquid phase to be deposited, which are then preferably deposited under pressure on the surface. The portions preferably have a volume in a range of 0.1 to 500 nl, particularly preferably in a range of 10 to 100 nl. The temperature of the surface to be coated should preferably be in a range of 30 to 60° C. The temperature of the liquid phase to be applied should preferably be in a range of 20 to 60° C., particularly preferably in a range of 20 to 35° C. The liquid phase is preferably a liquid or powdery coating material that i being applied to objects in a thin layer and is made-up by chemical or physical processes, such as, for example, evaporation of the solvent to form a continuous, solid film. The liquid phase usually consists of binding agents, pigments, solvents, fillers and additives, whereby the individual components can be used optionally. Liquid phases of said composition are often referred to as varnishes. Any binding agents known for the purpose of depositing coatings can be used as binding agent. Polymers to be described later are preferred binding agents. Any pigment suitable for the coating process can be used as pigments. Likewise, all solvents, fillers and additives suitable for the coating process can be used. During the deposition of the liquid phase in the form of a liquid solution or dispersion, the surface can be contacted to the liquid phase completely or partially. Depending on how small the amounts of liquid phase are selected for spraying or injecting, very fine patterns of the coating can be applied to the inner surface of the housing. The process of depositing also allows parts of the housing that should not have any coating to be prevented from contacting the coating. The process of dipping usually necessitates covering the parts that are not to be wetted.

The liquid phase, for example in the form of a liquid varnish, to be used for depositing on the surface should preferably have a viscosity in a range of 50 to 400 mPas (milli Pascal seconds), more preferably in a range of 50 to 200 mPas. The density of the liquid phase to be deposited should be in a range of 0.5 to 3 g/cm³, preferably in a range of 0.8 to 1.9 g/cm³. Preferably, the liquid phase to be deposited has a solids content in a range of 10 to 80 wt.-%, preferably in a range of 20 to 50 wt.-%, relative to the total mass of the liquid phase.

Dipping involves, for example, that the surface to be coated is pulled through a bath of the liquid phase to be applied. Alternatively, the surface can be immersed into the liquid phase and removed again, as is the practice in dip-coating. Repeated dipping allows coating different in thickness to be attained. Moreover, the thickness of the coating depends on the choice of the liquid phase, and other parameters, such as temperature of the liquid phase or of the inner surface during the application process, as mentioned above.

In a preferred embodiment of the method, the liquid phase is applied through an application opening that is provided over the inner surface, whereby the liquid phase is applied to the surface in the form of droplets. The rate of drop application can be selected sufficiently high such that almost a beam of liquid phase is generated. If the liquid phase is applied in the form of droplets, the droplets can be applied to the surface of the housing next to each other such that the entire desired surface is wetted. In this way, a continuous film of liquid phase is obtained, which can then become cured to form the electrically insulating coating. The distribution of the droplets can be done either by moving the nozzle relative to the housing or by moving the housing relative to the nozzle. The droplets can preferably be arranged so close together that the liquid phase can coalesce at the border of the droplets and thus forms a continuous surface in the form of a layer. By means of applying droplets, it is feasible to optionally contact some areas of the inner surface of the housing to more or less of the coating. The thickness of the coating can be varied both through the size of the droplets or through the speed of the motion of the nozzle with respect to the housing. Thus, it is also feasible to provide no coating in some areas, if desired.

In a preferred embodiment, the application of the liquid phase proceeds through an application opening provided over the inner surface, whereby the application opening and the surface are interconnected by means of the liquid phase. Since the liquid phase becomes connected to the inner surface of the housing during the application of the liquid phase to the surface, the liquid phase can be prevented from tearing on the surface. What can be attained by this means is that a very homogeneous film can be applied to the surface. By connecting the application opening to the inner surface of the housing, it becomes feasible to apply the liquid phase in the form of lines to the inner surface of the housing.

The two methods mentioned above, both the application of droplets and the connected application, are also known as micro-dosing. It is a particular feature of micro-dosing that it allows to easily apply the coating of varying thickness to objects, like the inner surface of the housing in the present case. The application opening can take any shape and size. This can concern, for example, an application opening of a shape selected from the group consisting of round, oval, angular and star-shaped or combinations thereof.

The application opening can have a diameter of 10 μm to 1 mm, preferably of 100 μm to 0.5 mm. Moreover, the surface area of the application opening can be 10 μm² to 1 mm², preferably can be in a range of 0.01 mm² to 0.5 mm², particularly preferably in a range of 0.05 mm² to 0.25 mm². Preferably, the liquid phase is applied through the nozzle to the surface by means of a pressure in a range of 1,100 to 5,000 mbar, preferably in a range of 1,100 to 4,000 mbar, particularly preferably in a range of 1,100 to 3,000 mbar. In most cases, the pressure at the application device can be adjusted.

Printing methods can be used as a further variant of the application of the liquid phase. These are characterised by the transfer of liquid phase via a carrier. Preferably, the carrier is capable of taking up the liquid phase, at least in part, and of releasing it again upon contact to a further surface. For example, the tampon printing method provides a roller with the liquid phase to be applied, which is pressed to or rolled over the surface to be coated. Depending on the design of the nozzle and/or roll or roller as well as the viscosity and polarity of the liquid phase to be applied, layers differing in thickness can be applied to the desired surface.

According to the present invention, the liquid phase applied to the inner surface during the application contains a polymer. The concentration of polymer in the liquid phase, which can just as well be a mixture of multiple polymers, is selected appropriately such that the electrically insulating coating formed from it contains at least 30 wt.-%, preferably at least 40 wt.-%, particularly preferably at least 50 wt.-%, relative to the mass of the coating. All polymers that are solid at room temperature and/or body temperature can be used as polymer. Preferably, the electrically insulating coating contains synthetic polymers, such as polyethylene (PE), polypropylene (PP), polyvinyl chloride (PVC), polyester, such as polycarbonates (PC) or polyethylene terephthalate (PET), polystyrene (PS), polytetrafluoroethylene (PTFE), polymethyl methacrylate (PMMA), polyamides, polyimides, polyethylene glycol (PEG), silicones or combinations thereof. In a preferred embodiment of the method, the polymer is selected from the group consisting of acrylates, alkyd resin, polyester imide, polyamide-imide, and silicones or at least two thereof. In an embodiment of the invention, the polyester imide or the polyamide-imide be imidised by more than 70%, particularly preferably more than 85%.

After application of the liquid phase to the inner surface of the housing, the inner surface of the housing can be subjected to a drying process. This serves for quick and uniform drying of the electrically insulating coating. In a preferred embodiment of the method, the electrically insulating coating is formed by irradiation or convection or both. Forming the coating can involve a drying process that can be accelerated and optimised through various supportive measures. Said drying process can, for example, proceed at a temperature in a range of 25 to 200° C., preferably in a range of 40 to 150° C., particularly preferably in a range of 50 to 80° C. The temperature during the drying process can be varied freely within these ranges.

Alternatively, or in addition to the elevated temperature, the liquid phase can also be subjected to an irradiation with electromagnetic waves to aid the formation of the layer. This radiation can be from the entire available wavelength range. Preferably, irradiation in the ultraviolet or infrared wavelength range is used, i.e. at wavelengths in a range of 800 to 2,000 nm and/or in a range of 200 to 400 nm, preferably in a range of 800 to 1,500 nm and/or in a range of 300 to 400 nm.

Using convection, in the form of, for example, elevated temperature or irradiation, the solvent of the liquid phase is evaporated faster than without these measures. As a result, only the solid components remain on the inner surface in the form of the coating. Moreover, applying radiation or elevated temperature allows, in addition, cross-linking reactions to be triggered in the polymers during the drying process. What this attains is that the coating is formed to be particularly hard and uniform. The coating is preferably a continuous layer of solid components. One property of the electrically insulating coating is that it forms a continuous layer, which can withstand even strong forces acting on the coated surface. Accordingly, strong shear and friction forces can be applied without damage to the coating. Getting damaged shall be understood to mean both a change in the thickness and the shape of the coating and, in particular, in the function of the coating. According to the invention, a coating did not get damaged, if it lost less than 10% of its initial breakdown voltage after the intervention or at the end of the service life of the device.

Using the application methods described above, coatings with a thickness in a range between 1 and 100 μm, preferably in a range between 10 and 80 μm, particularly preferably in a range between 30 and 60 μm can be attained. It is also conceivable to implement a combination of the deposition and dipping methods. Furthermore, two or more coating processes can be implemented. Accordingly, coating processes using different coating materials can proceed sequentially or simultaneously.

It is preferred to subject at least part of the inner surface of the housing to a chemical cleaning process before application of the coating. What said cleaning process can attain is that at least the inner surface assumes a texture that allows it is connect to the coating more easily and/or more lastingly or more firmly. Accordingly, the surface of the inner surface can be made either smooth or rougher, depending on which kind of substrate the selected coating forms better. During the cleaning, the surface of the inner surface can be made more porous, for example, such that the liquid phase can adhere better to the inner surface upon application of the coating. The cleaning can, for example, form cavities in the inner surface, into which the liquid phase can penetrate during application. In this manner, very intense bonding to the inner surface is attained while the inner surface is cured. A chemical cleaning can be implemented using all chemicals available for such purposes. Preferably, the chemical cleaning process is selected from the group consisting of: hot alkaline cleaning, rinsing with organic solvents, etching or at least two thereof. Hot alkaline cleaning can involve the use of an alkaline solution selected from the group consisting of sodium hydroxide (NaOH), potassium hydroxide (KOH), potassium phosphate (K₃PO₄) and hydrogen phosphates or any combination of at least two thereof. The concentration of the alkaline solution can preferably be in a range of 0.1 to 5 vol.-%, preferably in a range of 1 to 5 vol.-%, particularly preferably in a range of 2 to 3 vol.-%. The various alkaline solutions can, in addition, have surfactants, in particular anionic, cationic, or non-ionic surfactants, or a combination thereof admixed to them. For example acetone, alcohols or hydrocarbons can be used as organic solvents. Preferably, ethanol or isopropanol or mixtures thereof can be used as alcohols. The alcohols can be mixed with water in various concentrations. The alcohols or mixtures of alcohols are preferably used in a concentration in a range of 70 to 100 vol.-%, relative to the total solution. Alternatively or in addition, acids can be used for etching as well. Suitable etching agents to be mentioned include mainly hydrofluoric acid (HF), ammoniumbifluoride (NH₄HF₂), hydrochloric acid (HCl), nitric acid (HNO₃) and sulfuric acid (H₂SO₄) or combinations thereof. These various cleaning agents can also be used together or sequentially in any order.

Alternatively or additionally, a mechanical cleaning process can be applied as well. It has the same goal as the chemical cleaning. In a preferred method, at least part of the inner surface of the housing is subjected to a mechanical cleaning process before application of the coating. Similar to the chemical cleaning process, this can be used to increase or decrease the roughness of the surface, depending on what is to be achieved. In a preferred method, the mechanical cleaning process is selected from the group consisting of: plasma, sand blasting and glass bead cleaning or a combination of at least two thereof. Plasma cleaning can preferably be carried out with an oxygen/argon plasma. It is preferred in sand-blasting to use Al₂O₃ of an average grain size d₅₀ in a range of 50 to 200 μm, which acts on the surface with a pressure in a range of 1.5 to 5 bar. Glass bead cleaning utilises glass beads of a size in a range of 50 to 200 μm.

Another aspect of the invention describes a device that can be obtained according to the method described above.

A further aspect of the invention is a method for implanting a device according to the previous description, including the steps of:
providing the device;
opening a tissue;
introducing the device into the opened tissue;
closing the tissue, if applicable.

All variants of the embodiments of the device according to the invention are also applicable to the method according to the invention for implantation of a device. The apparatus can be provided in any usable form for the method. The step of providing the device can, for example, provide for the introduction of the device into an applicator, by means of which the device can be be introduced into the tissue by the user. Said applicator opens the tissue by itself during the application and introduces the device into the opened tissue. After removal of the applicator, the tissue can be re-closed, for example, by means of a patch, if necessary. Alternatively, the steps can also be performed manually. Thus, the device can be provided in its original usable and functional form. It is also feasible to provide the device in a sterile package that can be opened before use of the device. The opening of the tissue can be effected, for example, using a knife-like object, for example, a scalpel. Also, the introduction of the device into the opened tissue can be effected manually, just like the possible closing.

Furthermore, the information provided with respect to the device according to the invention apply as well to the method for generating a device and its product as well as the method for implanting the device according to the invention. This applies in particular to materials and spatial configurations.

Further details and features of the invention will become apparent from the following description of preferred embodiments, in particular in combination with the sub-claims. In this context, the individual features can be implemented alone or in combination of multiple of these features. The invention shall not be limited to the exemplary embodiments. The embodiments are shown schematically in the figures. The same reference numbers in individual figures denote identical or functionally identical or corresponding elements in terms of their functions.

### IN THE FIGURES

FIG. 1: Schematic structure of an implantable device in a longitudinal section;

FIG. 2: Schematic structure of an application device for the coating;

FIG. 3: Diagram showing the steps of the method for manufacturing a device according to the invention;

FIG. 4: Diagram showing the steps of the method for implanting a device according to the invention.

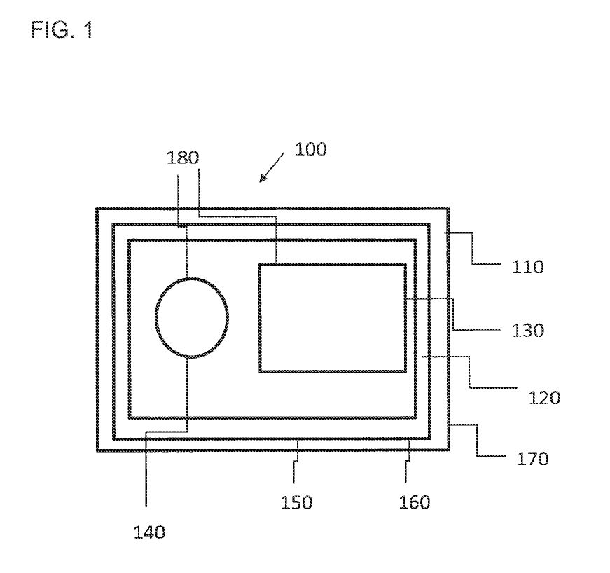

In FIG. 1, an implantable device 100 is shown schematically. The device 100 comprises a housing 110 having an inner surface 160 and an outer surface 170. The inner surface 160 of the housing 110 is connected to an electronically insulating coating 120 by means of the coating surface 150. Together with the electronic unit 180, which is composed of a battery 140 and a capacitor 130, said device is a device according to the invention. The coating 120 in this example consists of an alkyd resin containing 45 wt.-% of the polymer. The thickness of the electronically insulating coating in this context is 55 μm. Moreover, the device 100 can also comprise an electrode, which is not shown here, and further electronic components such as, for example, a storage unit or a processor.

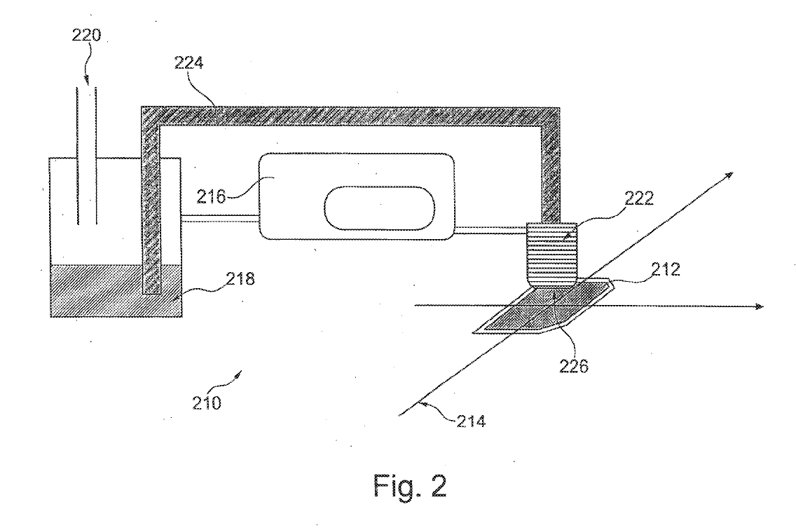

FIG. 2 shows a device 210 for applying a liquid phase 218 to a substrate 212. The substrate 212 is, in this case, part of the inner surface 160 of FIG. 1. A nozzle 222 is arranged appropriately with respect to the surface of the substrate 212 such that the liquid phase 218 can be applied appropriately such that a film of liquid can remain between the surface of substrate 212 and the nozzle. However, alternatively, the distance of the nozzle 222 to the substrate 212 can also be selected such that the liquid phase 218 can be applied in the form of droplets to the substrate. A homogeneous application of the liquid phase 218 by means of said device 210 is feasible because the substrate 212 is mounted such as to be movable back and forth along the reference coordinates 214. This allows, for example, the liquid phase 218 to be applied to the substrate 212 in the form of lines. Alternatively, the nozzle 222 can also be arranged to be movable along said reference coordinates 214. Using a control unit 216, the supply of the liquid phase 218 through the supply tube 224 to the nozzle 222 can be controlled. To vary the pressure of the liquid phase 218 within the supply tube 224, pressure can be applied to the liquid phase 218 by means of a pressure application 220. In the present example, the liquid phase 218 consists of the following components: 55 vol.-% of the solvent, Naphta (CAS#64742-48-9), 45 vol.-% of an alkyd resin. The varnish is Elmotherm FS 190 made by Elantas GmbH, Germany. The liquid phase is applied to the substrate 212 through a nozzle 222 having an application opening 226 having an opening diameter of 0.3 mm.

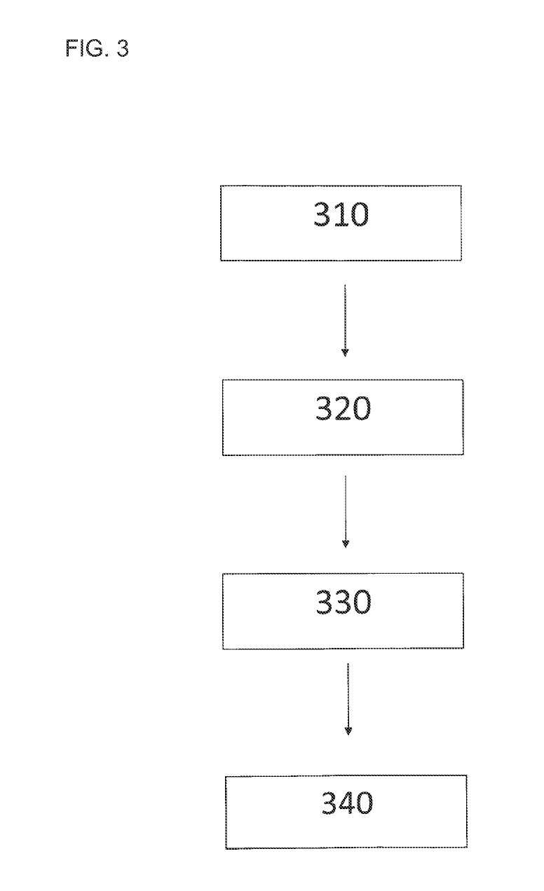

FIG. 3 schematically shows the flow of the method for manufacturing a device 100 according to the invention. This method is used, for example, for the manufacture of a device 100 shown in FIG. 1. Firstly, two housing parts are provided, which each were coated on their inside. The device of FIG. 2 referred to as application device 210 hereinafter was used for this purpose. In the first step 310, i.e. the providing of a housing 110, the housing 110 is positioned and fixed appropriately such that at least the inner surface 160 of the housing is accessible by means of application aids, such as nozzles or valves, as is shown, for example, in FIG. 2, whereby the nozzle 222 represents the application aid. The second step 320, i.e the applying of the electrically insulating coating 120, can proceed by means of the nozzle 222. A liquid phase 218, as previously described, can be used in this context. In a third step 330, which is optional and not obligatory, the applied liquid phase is dried. In the present example, said drying took place in an oven made by at Nabertherm M60/85HA GmbH at a temperature of 80° C. for one hour. After cooling of the housing at room temperature, the thickness of the electrically insulating coating 120 was 80 μm +/−2 μm, measured with a Mitutoyo micrometer screw. Then, the breakdown voltage of the housing 110 was measured. The breakdown voltage was 7 kV. The breakdown voltage of a sample, i.e. of a cardiac pacemaker in the present case, was measured by means of the following procedure:
1. The housing and the coating were contacted to electrodes of a WGHP601 potentiometer made by HCK GmbH, Essen, which is also referred to as “contacting”.
2. The smallest possible cut-off current was chosen, whereby the cut-off current was in a range <30 μA.
3. The voltage was increased slowly by hand using the potentiometer up to the breakdown voltage. The breakdown voltage is reached when the current exceeds the cut-off current of 30 μA. In this case, the breakdown voltage was 7 kV.
4. Then, 2 lower voltages allowing a test duration of 60 sec to be attained without reaching the cut-of current were preset. The selected voltages were 6 kV and 6.5 kV.

Moreover, the potentiometer is capable of determining the so-called cut-off current. This is defined as the last measured current determined by the potentiometer before the measurement was shut-off. In this particular example, the cut-off current was 17 μA. Subsequently, the electronic device 180 was introduced into one of the two housing halves of the housing 110. In the present case, the electronic unit 180 was a battery 140 and a capacitor 130, and an electrode. Finally, the two halves of the housing 110 were glued to each other.

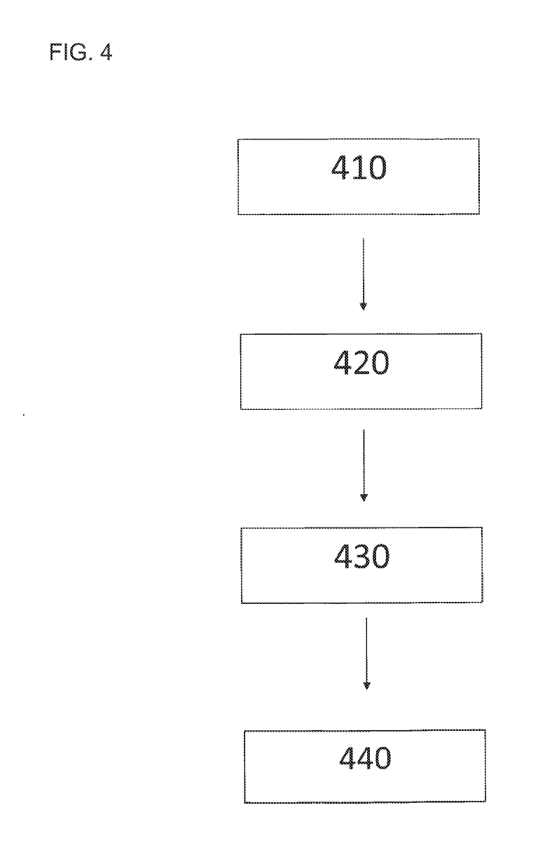

FIG. 4 schematically shows the flow of the method for implanting a device 100 according to the invention. In this context, the first step 410 is the provision of the housing 110. This can proceed as described previously in the form of a packaged device 110 or in the form of a device 100 that is introduced into an applicator. In the second step 420, the tissue is being opened. This can be accomplished by tools such as a knife or scalpel or an applicator. In the third step 430, the device 100 is introduced into the tissue. If this concerns a pacemaker or a defibrillator, for example an electrode can be connected to the heart and be controlled by the electronic unit 180, consisting of the capacitor 130 and the battery 140. However, the device 100 can just as well be a purely diagnostic device, such as a monitoring system of body functions, such as in the blood. Subsequently, the body can be closed again in a fourth step 440 of closing, if required. The closing can be done for example by applying a patch or a clamp.

### LIST OF REFERENCE NUMBERS

100 Implantable device
110 Housing
120 Electrically insulating coating
130 Capacitor
140 Battery
150 Coating surface
160 Inner surface
170 Outer surface
180 Electronic unit
210 Device for application
212 Substrate
214 Reference coordinates
216 Control unit
218 Liquid phase
220 Printing application
222 Nozzle
224 Supply tube
226 Application opening
310 1st step Providing the housing
320 2nd step Application
330 3rd step Drying
340 4th step Introducing el. unit
410 1st step Providing the device
420 2nd step Opening
430 3rd step Introduction into tissue
440 4th step Closing

1-20. (canceled)

21. A device comprising:
a housing having an inner surface and an outer surface; and
an electronic unit;
whereby the housing surrounds the electronic unit at least in part;
whereby at least a part of the inner surface of the housing comprises an electrically insulating coating that contains at least 30 weight-% of a polymer and has a coating surface facing the inner surface; and
whereby the inner surface and the coating surface are interconnected.

22. The device according to claim 21, whereby the electronic unit comprises a capacitor.

23. The device according to claim 22, whereby the capacitor has a capacitance in a range of 50 to 1000 μF.

24. The device according to claim 21, whereby the housing contains at least 30 weight-% titanium, relative to the housing.

25. The device according to claim 21, whereby the polymer is selected from the group consisting of acrylates, alkyd resin, polyester imide, polyamide-imide and silicones or at least two thereof.

26. The device according to claim 21, whereby the coating has a thickness in a range of 1 to 100 nm.

27. The device according to claim 21, whereby the insulating coating has a surface area in a range of 1 to 30 cm².

28. The device according to claim 21, whereby the breakdown voltage through the insulating coating is at least 2 kV.

29. A method for manufacturing a device comprising:
providing a housing having an inner surface and an outer surface;
applying an electrically insulating coating that contains at least 30 weight-% polymer and is made of a liquid phase to at least a part of the inner surface; and
introducing an electronic unit into the housing, whereby the housing surrounds the electronic unit at least in part.

30. The method according to claim 29, whereby the application of the electrically insulating coating proceeds by a process selected from the group consisting of deposition or dipping or a combination thereof.

31. The method according to claim 29, whereby the liquid phase is applied through an application opening that is provided over the inner surface, whereby the liquid phase is applied to the surface in the form of droplets.

32. The method according to claim 29, whereby the liquid phase is applied through an application opening that is provided over the inner surface, whereby the application opening and the surface are interconnected by means of the liquid phase.

33. The method according to claim 29, whereby the polymer is selected from the group consisting of acrylates, alkyd resin, polyester imide, polyamide-imide and silicones or at least two thereof.

34. The method according to claim 29, whereby the electrically insulating coating is formed by irradiation or convection or both.

35. The method according to claim 29, whereby at least part of the inner surface of the housing is subjected to a chemical cleaning process before application of the coating.

36. The method according to claim 35, whereby the chemical cleaning process is selected from the group consisting of: hot alkaline cleaning, rinsing with organic solvents, and etching or at least two thereof.

37. The method according to any one of the claim 29, whereby at least part of the inner surface of the housing is subjected to a mechanical cleaning process before application of the coating.

38. The method according to claim 37, whereby the mechanical cleaning process is selected from the group consisting of: plasma, sand blasting and glass bead cleaning or a combination of at least two thereof.

39. A method for implanting a device comprising:
providing the device, the device comprising a housing having an inner surface and an outer surface and an electronic unit, wherein the housing surrounds the electronic unit at least in part, wherein at least a part of the inner surface of the housing comprises an electrically insulating coating that contains at least 30 weight-% of a polymer and has a coating surface facing the inner surface, and wherein the inner surface and the coating surface are interconnected;
opening a tissue;
introducing the device into the opened tissue;
closing the tissue, if applicable.
